(19) United States
(12) Patent Application Publication
(10) Pub. No.: US 2025/0051470 A1
(43) Pub. Date: Feb. 13, 2025

(54) METHODS OF TREATING GRAFT VERSUS HOST DISEASE

(71) Applicant: ADIENNE S.A., Lugano (CH)

(72) Inventor: Antonio Francesco DI NARO, Morcote (CH)

(21) Appl. No.: 18/507,904

(22) PCT Filed: May 13, 2022
(86) PCT No.: PCT/IB2022/054501

§ 371 Date: Nov. 13, 2023

Related U.S. Application Data
(60) Provisional application No. 63/201,805, filed on May 13, 2021.

(51) Int. Cl. C07K 16/28

(52) U.S. Cl. CPC C07K 16/2896; C07K 2317/21; C07K 2317/24; C07K 2317/35

(57) ABSTRACT

This disclosure relates to methods of using an anti-CD26 antibody and antigen binding fragments thereof for the treat-ment of graft versus host disease.

### FIELD

This disclosure relates to methods of using an anti-CD26 antibody and antigen binding fragments thereof for the treatment of Graft versus Host Disease (GvHD).

### BACKGROUND

Acute GvHD is the major complication of allogeneic hematopoietic stem cell transplantation (HSCT) (J Bolaños-Meade et al., 2004). Acute GvHD produces significant mortality, morbidity and complicates patient management, resulting in organ toxicity, frequent infections, malnutrition and substantial delay in recovery from transplantation. Acute GvHD usually occurs within the first 3-4 months of allogeneic HSCT and may involve the skin, liver and intestinal tract. It is believed that T-lymphocytes contained in the donor graft respond in vivo to disparate major (HLA) or minor (non-HLA) histocompatibility antigens expressed by recipient tissues, initiating a cascade of events leading to the signs and symptoms of acute GvHD.

The syndrome of acute GvHD includes the following signs and symptoms: skin involvement (maculopapular exanthema) is usually the first sign. Lesions may be pruritic or painful, red to violaceous in color, and often involve the palms and soles. Acute GvHD of the gut may involve the stomach, small bowel and colon producing persistent nausea and vomiting or profuse diarrhea, intestinal bleeding, cramping, abdominal pain and paralytic ileus. Liver GvHD produces cholestatic liver injury with hyperbilirubinemia and, in some cases, hepatocellular enzyme elevations.

The use of cyclosporine (CSA) or tacrolimus (FK) plus short course methotrexate (MTX) has lowered the risk for acute GvHD compared to single agent or other combination therapy, but even with these regimens, 35-50% of patients develop Grade II-IV acute GvHD (J Bolaños-Meade et al., 2004; H J Deeg, 2007; A. Bacigalupo, 2007). Older recipients or those with unrelated or partially HLA-matched donors may develop more frequent or possibly more therapy-resistant acute GvHD.

Response to initial treatment is a key predictor of clinical outcome. Mortality in patients with grade II-IV acute GvHD is greatest among those who fail to achieve a complete response to initial treatment (P J Martin, et al., 1990; D Weisdorf, et al., 1990; M L MacMillan et al., 2002; J Bolaños-Meade et al., 2005). A very early response, as evidenced by the ability to begin a steroid taper on Day 5 of therapy is also associated with a favorable prognosis (M T Van Lint, et al., 2006). It is possible that early responders do better, at least in part, because the steroid taper begins sooner and reduces the risk of complicating opportunistic infections, a common cause of disease. In general, cutaneous disease responds promptly but lower gastro-intestinal and hepatic involvement responds less well to therapy (M L MacMillan et al., 2002). Other risks for failure of initial therapy include early onset of GvHD, increasing HLA disparity and older age (P J Martin, et al., 1990; ML MacMillan et al., 2002).

The suboptimal response and long-term survival associated with corticosteroids alone has prompted investigation of additional immunosuppressive agents in the initial therapy of acute GvHD. Unfortunately, this strategy has to date not yielded superior outcomes compared to steroids alone. In some cases, the benefit of better initial GvHD control with an additional agent is offset by a higher risk of infection or other side effects. The most widely studied agent in this context is anti-thymocyte globulin (ATG). Single arm Phase II studies of prednisone+ATG as initial therapy of acute GvHD have reported response rates from 67% to 80% (M J Dugan, et al., 1997; F Graziani, et al, 2001). However, in a randomized trial of prednisolone±equine ATG, there was no difference in response rates or survival, but infectious morbidity, especially CMV disease and pneumonitis, was higher in the combined immunosuppression group (L Cragg, et al., 2000).

Despite continued advances in the field of GvHD research, there remains a significant unmet medical need in the treatment of acute GvHD, as 35-50% of patients undergoing allogeneic hematopoietic stem cell transplantation develop Grade II-IV acute GvHD (J Bolaños-Meade et al., 2004; H J Deeg, 2007; A. Bacigalupo, 2007). To date, the primary treatment option for acute GvHD remains systemic administration of corticosteroids in addition to standard immunosuppression protocols.

### SUMMARY

The present disclosure is directed to a method of treating graft-versus-host disease (GvHD) in a subject, comprising administering a therapeutically effective amount of an anti-CD26 antibody to the subject, wherein the antibody is administered at a dose of between 4.0 mg/m²/day and 25.0 mg/m²/day, and wherein the antibody is administered once daily for five days followed by administration every other day for a total of 16 doses.

In one aspect, the anti-CD26 antibody is a full-length antibody. In another aspect, the antibody is a monoclonal, human, humanized, chimeric, multivalent antibody, or an antigen-binding fragment thereof. In another aspect, the antibody has an isotype selected from the group consisting of IgG1, IgG2, IgG3, IgG4, IgM, IgA, IgD and IgE. In another aspect, the antibody has an IgG2b isotype.

In one aspect, the anti-CD26 antibody is begelomab, 1F7, or CM03. In another aspect, the anti-CD26 antibody is produced in Chinese hamster ovary (CHO) cells. In another aspect, the anti-CD26 antibody is produced from a hybridoma cell line deposited at CBA-ICLC of Genoa (Italy) as deposit number PD 12002.

In one aspect, the anti-CD26 antibody comprises
(a) a heavy chain variable region CDR1 comprising the sequence set forth in SEQ ID NO:7;
(b) a heavy chain variable region CDR2 comprising the sequence set forth in SEQ ID NO:8;
(c) a heavy chain variable region CDR3 comprising the sequence set forth in SEQ ID NO:9;
(d) a light chain variable region CDR1 comprising the sequence set forth in SEQ ID NO:10;
(e) a light chain variable region CDR2 comprising the sequence set forth in SEQ ID NO:11; and
(f) a light chain variable region CDR3 comprising the sequence set forth in SEQ ID NO:12. In another aspect, the anti-CD26 antibody comprises heavy and light chain variable regions comprising the sequences set forth in SEQ ID NOs:3 and 5, respectively. In another aspect, the anti-CD26 antibody comprises heavy and light chains constant regions comprising the sequences set forth in SEQ ID NOs:1 and 2, respectively.

In one aspect, the anti-CD26 antibody is administered at a dose of 4, 5, 6, 7, 8, 9, 10, 11, 12, 13, 14, 15, 16, 17, 18, 19, 20, 21, 22, 23, 24, or 25 mg/m². In another aspect, the anti-CD26 antibody is administered at a dose of 16 mg/m².

### DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.

### Definitions

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system. In one embodiment, an antibody is an intact antibody.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” includes monospecific, bispecific, multivalent or multi-specific, antibodies, as well as a single chain antibody.

As used herein, an “IgG antibody” has the structure of a naturally occurring IgG antibody, i.e., it has the same number of heavy and light chains and disulfide bonds as a naturally occurring IgG antibody of the same subclass. For example, an anti-CD26 IgG1, IgG2, IgG3 or IgG4 antibody consists of two heavy chains (HCs) and two light chains (LCs), wherein the two heavy chains and light chains are linked by the same number and location of disulfide bridges that occur in naturally occurring IgG1, IgG2, IgG3 and IgG4 antibodies, respectively (unless the antibody has been mutated to modify the disulfide bonds)

An “isolated antibody” refers to an antibody that is substantially free of other antibodies having different antigenic specificities (e.g., an isolated antibody that binds specifically to CD26 is substantially free of antibodies that bind specifically to antigens other than CD26). An isolated antibody that binds specifically to CD26 may, however, have cross-reactivity to other antigens, such as CD26 molecules from different species. Moreover, an isolated antibody may be substantially free of other cellular material and/or chemicals.

The antibody may be an antibody that has been altered (e.g., by mutation, deletion, substitution, conjugation to a non-antibody moiety). For example, an antibody may include one or more variant amino acids (compared to a naturally occurring antibody) which change a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs may be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-CD26 antibody binds specifically to CD26.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments or portions of a full-length antibody. Examples of binding fragments encompassed within the term “antigen-binding portion” or “antigen-binding fragment” of an antibody, e.g., an anti-CD26 antibody described herein, include:
(1) a Fab fragment (fragment from papain cleavage) or a similar monovalent fragment consisting of the VL, VH, LC and CH1 domains;
(2) a F(ab′)2 fragment (fragment from pepsin cleavage) or a similar bivalent fragment comprising two Fab fragments linked by a disulfide bridge at the hinge region;
(3) a Fd fragment consisting of the VH and CH1 domains;
(4) a Fv fragment consisting of the VL and VH domains of a single arm of an antibody,
(5) a single domain antibody (dAb) fragment (Ward et al., (1989) Nature 341:544-46), which consists of a VH domain;
(6) a bi-single domain antibody which consists of two VH domains linked by a hinge (dual-affinity re-targeting antibodies (DARTs));
(7) a dual variable domain immunoglobulin;
(8) an isolated complementarity determining region (CDR); and
(9) a combination of two or more isolated CDRs, which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. In some embodiments, an antibody is an antigen-binding fragment.

The term “CD26” refers to dipeptidyl peptidase 4 (DPP4). The terms CD26 and DPP4 are used interchangeably herein. The term “CD26” includes variants, isoforms, homologs, orthologs and paralogs. For example, antibodies specific for a human CD26 protein may, in certain cases, cross-react with a CD26 protein from a species other than human. In other embodiments, the antibodies specific for a human CD26 protein may be completely specific for the human CD26 protein and may not exhibit species or other types of cross-reactivity, or may cross-react with CD26 from certain other species, but not all other species (e.g., cross-react with monkey CD26 but not mouse CD26). The term “human CD26” refers to human sequence CD26, such as the complete amino acid sequence of human CD26 having GenBank Accession No. AH005372.3. The human CD26 sequence may differ from human CD26 of GenBank Accession No. AH005372.3 by having, e.g., conserved mutations or mutations in non-conserved regions and the CD26 has substantially the same biological function as the human CD26 of GenBank Accession No. AH005372.3.

A particular human CD26 sequence will generally be at least 90% identical in amino acid sequence to human CD26 of GenBank Accession No. AH005372.3 and contains amino acid residues that identify the amino acid sequence as being human when compared to CD26 amino acid sequences of other species (e.g., murine). In certain cases, a human CD26 can be at least 95%, or even at least 96%, 97%, 98%, or 99% identical in amino acid sequence to CD26 of GenBank Accession No. AH005372.3. In certain embodiments, a human CD26 sequence will display no more than 10 amino acid differences from the CD26 sequence of GenBank Accession No. AH005372.3. In certain embodiments, the human CD26 can display no more than 5, or even no more than 4, 3, 2, or 1 amino acid difference from the CD26 sequence of GenBank Accession No. AH005372.3.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction=0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity. An alignment for purposes of determining percent nucleic acid sequence identity can be carried out according to procedures known in the art, as described for example in EP 1 241 179 B1. For example, a skilled person can use publicly available computer software, such as using publicly available computer software such as BLAST, BLAST-2, ALIGN or Megalign (DNASTAR) software. A skilled person can determine appropriate parameters for measuring alignment, including any algorithms needed to achieve maximal alignment over the full length of the sequences subjected to comparison. According to a preferred embodiment, the % identity values can be generated using the WU-BLAST-2 computer program. According to a preferred embodiment, the following computer program and parameters are used: The identity values used herein are generated by the BLASTN module of WU-BLAST-2 set to the default parameters, with overlap span and overlap fraction set to 1 and 0.125, respectively. A % nucleic acid sequence identity value can be obtained by dividing (a) the number of matching identical nucleotides between a particular nucleic acid sequence as set forth herein which is subjected to comparison (e.g. a particular nucleic acid sequence characterized by a sequence identifier in the sequence listings), and the comparison nucleic acid molecule of interest to be compared, for example the number of matching identical nucleotides as determined by WU-BLAST-2, by (b) the total number of nucleotide residues of the particular nucleic acid sequence as set forth herein which is subjected to comparison (e.g. a particular nucleic acid sequence characterized by a sequence identifier in the sequence listings).

A “patient” as used herein includes any patient who is afflicted with GvHD. The terms “subject” and “patient” are used interchangeably herein.

“Administering” refers to the physical introduction of a composition comprising a therapeutic agent to a subject, using any of the various methods and delivery systems known to those skilled in the art. Routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, in some embodiments, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system.

In one example, an “effective amount” is the amount of anti-CD26 antibody clinically proven to affect a significant decrease in the inflammatory response of GvHD. As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore not provided as a mg/m² dose, but rather as an absolute amount of the agent (e.g., the anti-CD26 antibody). For example, a 60 kg person and a 100 kg person would receive the same dose of the composition (e.g., 100 mg of an anti-CD26 antibody).

The term “body surface area based dose” as referred to herein means that a dose that is administered to a patient is calculated based on the surface area of the patient. For example, when a patient with 2.0 body surface area (BSA) requires 4 mg/m² of an anti-CD26 antibody, one can draw the appropriate amounts of the anti-CD26 antibody (i.e., 8 mg).

“Dosing interval,” as used herein, means the amount of time that elapses between doses of the anti-CD26 antibody disclosed herein being administered to a subject. Dosing interval can thus be indicated as ranges.

The term “dosing frequency” as used herein refers to the frequency of administering doses of the anti-CD26 antibody disclosed herein in a given time. Dosing frequency can be indicated as the number of doses per a given time, e.g., once a week or once in two weeks.

The terms “about once a week,” “once about every week,” “once about every two weeks,” or any other similar dosing interval terms as used herein means approximate number, and “about once a week” or “once about every week” can include every seven days±two days, i.e., every five days to every nine days. The dosing frequency of “once a week” thus can be every five days, every six days, every seven days, every eight days, or every nine days. “Once about every two weeks” can include every fourteen days±three days, i.e., every eleven days to every seventeen days. Similar approximations apply, for example, to once about every three weeks, once about every four weeks, once about every five weeks, once about every six weeks and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The term “CD26 positive” or “CD26 expression positive,” relating to CD26 expression, refers to the proportion of cells in a test tissue sample, typically comprising muscle cells, which the tissue sample is scored as expressing CD26

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

Various aspects of the invention are described in further detail in the following subsections.

### CD26 Antibodies

In one aspect, the invention features methods of using an anti-CD26 antibody in the treatment of an GvHD. Anti-human-CD26 antibodies (or VH/VL domains derived therefrom) suitable for use in the invention can be generated using methods well known in the art. Alternatively, art recognized anti-CD26 antibodies can be used.

In some embodiments, the anti-CD26 antibody is produced from the hybridoma cell line deposited on Sep. 11, 2012 under the Budapest Treaty at the Centro di Biotecnologie Avanzate (CBA)-Interlab Cell Line Collection (ICLC) of Genoa (L. go R. Benzi, 10, Genoa, Italy) as deposit PD 12002. In another embodiment, the anti-CD26 antibody used in the methods of the invention bind the same epitope as an antibody produced by the hybridoma cell line deposited at CBA-ICLC of Genoa (Italy) deposited as PD 12002.

In some embodiments, the anti-CD26 antibody is begelomab comprising heavy and light chains comprising the sequences shown in SEQ ID NOs:1 and 2, respectively, or antigen binding fragments and variants thereof, as described in U.S. Pat. Nos. 9,376,498 and 10,208,126, the teachings of which are hereby incorporated by reference.

In other embodiments, the antibody has the heavy and light chain CDRs or variable regions of begelomab. Accordingly, in one embodiment, the antibody comprises CDR1, CDR2, and CDR3 domains of the VH region of begelomab having the sequence set forth in SEQ ID NO:3, and CDR1, CDR2 and CDR3 domains of the VL region of begelomab having the sequence set forth in SEQ ID NO:5. In another embodiment, the antibody comprises CDR1, CDR2 and CDR3 domains comprising the sequences set forth in SEQ ID NOs:7, 8, and 9, respectively, and CDR1, CDR2 and CDR3 domains comprising the sequences set forth in SEQ ID NOs:10, 11, and 12, respectively. In another embodiment, the antibody comprises VH and/or VL regions comprising the amino acid sequences set forth in SEQ ID NO:3 and/or SEQ ID NO:5, respectively. In another embodiment, the antibody comprises heavy chain variable (VH) and/or light chain variable (VL) regions encoded by the nucleic acid sequences set forth in SEQ ID NO:4 and/or SEQ ID NO:6, respectively. In another embodiment, the antibody competes for binding with and/or binds to the same epitope on CD26 as the above-mentioned antibodies. In another embodiment, the antibody has at least about 90% variable region amino acid sequence identity with the above-mentioned antibodies (e.g., at least about 90%, 95% or 99% variable region identity with SEQ ID NO:3 or SEQ ID NO:5).

In some embodiments, the anti-CD26 antibody or antigen-binding portion thereof cross-competes with begelomab for binding to human CD26. In other embodiments, the anti-CD26 antibody or antigen-binding portion thereof binds to the same epitope as begelomab.

In some embodiments, the anti-CD26 antibody is codon-optimized for expression in a host cell. In one embodiment, the anti-CD26 antibody is begelomab that is codon optimized for expression in Chinese Hamster Ovary (CHO) cells. In another embodiment, the codon optimized begelomab comprises the heavy chain and light chain of SEQ ID NOs:15 and 16, respectively.

In one embodiment, the anti-CD26 antibody is an antibody or antigen-binding fragment thereof described in U.S. Pat. No. 7,658,923 (for example, IF7); U.S. Pat. No. 7,462,698 (for example, CM03), U.S. Pat. No. 8,771,688, and EP Pat. No. 3 348 276 A1. Antibodies or antigen-binding fragments thereof that compete with any of the above-referenced art-recognized antibodies for binding to CD26 also can be used.

In certain embodiments, an anti-CD26 antibody is used to determine CD26 expression. In some embodiments, an anti-CD26 antibody is selected for its ability to bind to CD26 in formalin-fixed, paraffin-embedded (FFPE) tissue specimens. In other embodiments, an anti-CD26 antibody is capable of binding to CD26 in frozen tissues. In further embodiments, an anti-CD26 antibody is capable of distinguishing membrane bound, cytoplasmic, and/or soluble forms of CD26.

### Immunosuppressive Agents

In some embodiments, the methods of the invention feature using one or more immunosuppressive agents in combination with the anti-CD26 antibody to treat GvHD. In one embodiment, the immunosupressive agent is considered the standard of care for treatment of the GvHD. An “immunosupressive agent” is a compound that inhibits or prevents the activity of the immune system. In one embodiment, the immunosuppressive agent is a corticosteroid. Corticosteroids are well known in the art and can comprise in particular mineralocorticoids and glucocorticoids. Glucocorticoids can be anti-inflammatory agents. As used herein, the term corticosteroids can include steroids which can be in particular produced in the adrenal cortex of vertebrates, as well as can encompass synthetic corticosteroids or synthetic or natural corticosteroid analogs, including compounds that mimic the activity of natural steroid hormones, such as e.g. cortisone and hydrocortisone. Corticosteroid analogs may in particular encompass synthetic or natural chemical compounds which resemble in structure and/or function any of naturally occurring steroids elaborated by the adrenal cortex.

One or more corticosteroids can be selected from the group consisting of alclometasone dipropionate, amcinonide, amcinafel, amcinafide, beclamethasone, betamethasone, betamethasone dipropionate, betamethasone valerate, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone, diflorasone diacetate, dichlorisone, esters of betamethasone, fluazacort, flucetonide, flucloronide, fludrotisone, fluorocortisone, flumethasone, flunisolide, fluocinonide, fluocinolone, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, loteprendol, medrysone, meprednisone, methylprednisone, methylprednisolone, 6-methylprednisolone, mometasone furoate, paramethasone, paramethasone acetate, prednisone, prednisolone, prednidone, prednicarbate, triamcinolone acetonide, triamcinolone hexacatonide, tixocortol prednisolone, and triamcinolone, pharmaceutically acceptable salts thereof, derivatives thereof, and mixtures thereof. In other embodiments, the immunosuppressive agents include methotrexate, azathioprine, mycophenolate mofetil, alkylating agents such as cyclophosphamide, nitrosoureas, and platinum compounds, and monoclonal antibodies such as rituximab, tocilizumab, alemtuzumab, and saflimumab. In one embodiment, immunosuppressive agents include pharmaceutically acceptable salts, acids or derivatives of any of the above; as well as combinations of two or more of the above.

### Pharmaceutical Compositions

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-CD26 antibody). Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-CD26 antibody is administered intravenously.

### Methods of the invention

In some embodiments, the present disclosure is directed to a method investigate the pharmacokinetics, pharmacodynamics, safety and clinical activity of an anti-CD26 antibody (e.g., begelomab) as an initial treatment of acute GvHD in combination with standard steroid therapy. In some embodiments, the method is a dose escalation study designed to investigate the pharmacokinetics, pharmacodynamics, safety and clinical activity of an anti-CD26 antibody (e.g., begelomab) as an initial treatment of acute Graft- versus-Host Disease in combination with standard steroid therapy. In some embodiments, the objectives of the trial are: (1) to establish the optimal biological dose (OBD) of intravenously infused begelomab in terms of modulation of CD26/CD3 lymphocyte activity; (2) to investigate the pharmacokinetics and pharmacodynamics of begelomab following escalating multiple doses of begelomab; (3) to investigate efficacy endpoints;, and (4) to investigate the safety and tolerability of multiple doses of begelomab in patients with acute Graft-versus-Host Disease.

### EXAMPLES

### Overall Study Design and Plan Description

### Study Design

This is a Phase II, multi-center, open label, dose-escalation study in sequential cohorts to investigate the pharmacokinetics, pharmacodynamics, safety and clinical activity of begelomab as an initial treatment of acute Graft-versus-Host Disease in combination with standard steroid therapy.

The study will be divided in two phases. At initial stage, up to 6 patients per cohort will be enrolled into sequential cohorts receiving increasing multiple doses of begelomab; each subject will take part in one cohort only. The study is planned to comprise up to four dose levels; two additional dose levels may be added as needed. At the second stage, upon reaching the optimal biological dose (OBD), up to 24 additional subjects will be enrolled and treated at the OBD, as per the same treatment schedule as in the dose escalation phase.

The starting dose level will be 4.0 mg/m², while the dose increments for subsequent cohorts will be decided on the basis of emerging safety, tolerability and CD26+binding data. The decision to escalate the dose to the next higher level requires review of safety-, tolerability and pharmacodynamic data from at least four subjects that completed the full treatment period in the preceding group. Dose escalation will stop upon reaching generally accepted criteria for safety and tolerability as outlined in Section 6.2, the predefined criteria for the OBD or reaching the NOAEL exposure, whichever occurs first. The maximal dose level to be tested is 25.0 mg/m²/day.

For this study, the OBD is defined as the dose leading to a maximum receptor occupancy and yielding the most desirable clinical effects.

The total study duration per patient will be around 26 weeks, consisting of a screening period (Day −1 to Day 0), 27 days of treatment, and four post-treatment safety follow-up visits scheduled at Day 28−1/+2, Day 56±3, Day 100±7 and Day 180±7 (first day of treatment is defined as Day 1).

### Study Objectives

The objectives of the trial will be:
to establish the optimal biological dose of intravenously infused begelomab in terms of modulation of CD26/CD3 lymphocyte activity;
to investigate the pharmacokinetics and pharmacodynamics of begelomab following escalating multiple doses of begelomab;
to investigate efficacy endpoints;
to investigate the safety and tolerability of multiple doses of begelomab in patients with acute Graft-versus-Host Disease.

### Study Endpoints

### Primary Endpoint

The primary endpoint of the study will be the dose of begelomab leading to a maximum receptor occupancy within the tested dose range 4.0-25.0 mg/m²/day, as determined with flow cytometry.

### Secondary Endpoints

The secondary endpoints will be:
Cumulative overall response at Day 28, Day 56, Day 100 and Day 180.

Overall response is defined as graft-versus-host disease (GvHD) complete response (CR) or partial response (PR), defined as:
GvHD CR is complete resolution of all signs and symptoms of acute GvHD in all evaluable organs without intervening salvage;
GvHD PR is improvement in 1 or more organs involved with GvHD symptoms without progression in others.
Duration of overall response [time frame: from baseline to Day 180].

Defined as the time from first response until GvHD progression or death.
Overall response of grade II-IV visceral acute GvHD at Day +28, Day 56, Day 100 and Day 180.

Grade and stage of visceral acute GvHD will be determined according to the MAGIC criteria (A C Harris et al., 2015). A confirmatory biopsy is recommended when possible in term of clinical patient's status to establish the diagnosis of gastrointestinal GvHD.
Incidence and severity of systemic infections [time frame: Day 28, Day 100 and Day 180].
Number of participants that experienced at least one infection.
Overall Survival at Day 28, Day 56, Day 100 and Day 180 [time frame: from baseline to Day 180].

Proportion of participants who survive until Day 28, Day 56, Day 100 and Day 180.
GvHD relapse free survival at Day 100 and Day 180 [time frame: Day 100 and Day 180].
Transplant-related mortality at Days 28, 56, 100 and 180.
Cumulative dose of steroid treatment [time frame: Day 7, Day 15, Day 28, Day 56, Day 100, Day 180].

The cumulative steroid dose for each patient will be calculated (expressed in prednisone equivalents) by adding the doses in a cumulative manner.
Percentage reduction of the steroid dose compared with respect to initial dosing [time frame: Day 28, Day 56, Day 100, Day 180].
Incidence of steroid-resistant acute GvHD at Day 28, Day 56, Day 100 and Day 180.
Incidence of chronic GvHD at Day 100 and Day 180.
Grade and stage of chronic GvHD will be determined according to the NIH consensus criteria 2014 (M H Jagasia et al, 2014).
Pharmacokinetics of begelomab as determined by the Cₘₐₓ, tₘₐₓ, AUC₀₋ₜ, CL, t_{1/2λz}, and the accumulation ratios for Cₘₐₓ and AUC [time frame: from Day 1 to Day 31].
Incidence and titre time-course of human anti-murine antibody (HAMA) and its neutralizing ability [time frame: Days 1 (prior to start of begelomab), 5, 11, 17, 28, 56, 100 and 180].
Time course of circulating CD26+[time frame: from Day −1 to Day 180].
Adverse event (AE) and Serious adverse event (SAE) incidence [time frame: from baseline to Days 28, 100 Day 180].
Proportion of participants who have a treatment-related AE (TRAE) after infusion of begelomab [time frame: from baseline to Days 28, 100 and 180].
TRAE is defined as an event with a definite/certain, probable, or possible causality to study medication. This study will use the descriptions and grading scales from NCI CTCAE v4.0 for hematologic and non-hematologic toxicities.

Exploratory endpoints will be exploratory biomarkers [time frame: from baseline to Day 180]. If a patient agrees to participate in the optional exploratory biomarkers research part of this study, leftover biological samples that remain after the planned analyses are completed may be analyzed for further investigations, e.g., exploratory biomarkers to assess correlations with disease activity, effects of study drug, and clinical outcomes.

### Study Population

The study population will compromise patients higher than 12 years of age, irrespective of gender, who is diagnosed with acute GvHD host disease grades II-IV.

The study will be divided in two phases. At initial stage, up to 6 patients per cohort will be enrolled into sequential cohorts receiving increasing multiple doses of begelomab; each subject will take part in one cohort only. The study is planned to comprise up to four dose levels; two additional dose levels may be added as needed. At the second stage, upon reaching the optimal biological dose, up to 24 additional subjects will be enrolled and treated at the OBD, as per the same treatment schedule as in the dose escalation phase.

Only patients who have signed the informed consent form and meet all inclusion criteria and no exclusion criteria will be included in the trial.

### Inclusion Criteria

To be eligible for inclusion into this study, each patient must fulfill ALL of the following inclusion criteria:
Age≥12 years old.
Subject must be willing and able to comply with study requirements, remain at the clinic, and return to the clinic for the follow-up evaluation, as specified in this protocol during the study period.
Subject must be able and willing to provide signed informed consent. For adolescent patients, informed consent will be obtained from their parents or legal guardians.
Patients receiving all types of allogeneic transplantation except patients transplanted with ex vivo

T-cell-depleted grafts.
Patients with acute GvHD Grade II, III and IV as per MAGIC criteria, occurring after allogeneic hematopoietic transplant using either bone marrow (BM), peripheral blood stem cells (PBSC) or cord blood (CB), or after donor lymphocyte infusion (DLI).

A confirmatory biopsy is required, if clinically feasible, to confirm diagnosis of skin GvHD, and is recommended when feasible in case of gastrointestinal and liver involvement. Reasons for any omission of biopsy will be documented in the CRF.

If biopsy results are not available at the baseline visit, the treatment with begelomab can be started based on presumptive diagnosis when other etiologies are ruled out.
De novo acute GvHD requiring systemic therapy. GvHD is defined as the presence of skin rash and/or persistent nausea, vomiting, and/or diarrhea and/or cholestasis presenting in a context in which acute GvHD is likely to occur and where other aetiologies such as drug rash, enteric infection, or hepatotoxic syndromes are unlikely or have been ruled out.

A confirmatory biopsy is required, if clinically feasible, to confirm diagnosis of skin GvHD, and is recommended when feasible in case of gastrointestinal and liver involvement. Reasons for any omission of biopsy will be documented in the CRF.

If biopsy results are not available at the baseline visit, the treatment with begelomab can be started based on presumptive diagnosis when other etiologies are ruled out.
The patient must have had no previous systemic immune suppressive therapy for treatment of acute GvHD except for a maximum 72 hours of prior systemic corticosteroid therapy at ≤2.0 mg/kg methylprednisolone or equivalent after the onset of acute GvHD.
Evidence of myeloid engraftment (absolute neutrophil count (ANC)≥500/μL).
No evidence of relapse of underlying disease for which the patient required HSCT.
Patients with adequate renal reserve as defined by serum creatinine <3×upper limit of normal (ULN) or calculated creatinine clearance (CLcr) >30 mL/min using the Cockroft-Gault equation (1976).
Adequate pulmonary function as assessed by ≥92% oxygen saturation (SaO2) by pulse oximetry at rest.
Cardiac ejection fraction >40%.
Lansky performance score ≥70% for patients aged <16 years or Karnofsky performance score ≥50% for patients aged ≥16 years).
Life expectancy of greater than 1 month.
Women of child-bearing potential and men must agree to take adequate contraceptive measures in order to avoid any pregnancies of their sexual partners during the course of the study (or for at least 3 months following the last dose of study drug, whichever is longer). Acceptable methods of birth control include oral, injected or implanted hormonal methods of contraception, placement of an intrauterine device IUD) or intrauterine system (IUS), barrier methods of contraception: condom or occlusive cap (diaphragm or cervical/vault caps) with spermicidal foam/gel/film/cream/suppository. Abstinence is only considered an acceptable form of contraception when it is the usual life style of an individual.
Women must have a negative pregnancy test at Screening and at Baseline and must not be breastfeeding.

### Investigational Product

In this study, the Investigational Medicinal Product (IMP) will be begelomab, a murine monoclonal IgG2b antibody against CD26, produced in a hybridoma cell line. The starting dose level will be 4.0 mg/m²/day. In some aspects, the anti-CD26 antibody is administered at a dose of 4, 5, 6, 7, 8, 9, 10, 11, 12, 13, 14, 15, 16, 17, 18, 19, 20, 21, 22, 23, 24, or 25 mg/m²/day. In some aspects, the anti-CD26 antibody is administered at a dose of 16 mg/m²/day.

The investigational medicinal product will be administered as a 1-hour intravenous infusion into a central vein via a dedicated catheter (i.e. CVC or PICC) once daily for five days after enrolment (Days 1-5) followed by administration every other day for another eleven doses (Days 7, 9, 11, 13, 15, 17, 19, 21, 23, 25, 27) for a total of 16 doses. First day of treatment is defined as Day 1.

In each cohort, review of all available safety and tolerability data following the first five doses (Day 1-5) of the first subject will occur prior to enrolling the other subjects in the cohort. There will be no intra-cohort dose escalations.

Upon reaching the optimal biological dose, an additional cohort of up to 24 patients will be enrolled and treated at the OBD as above.

### Study Steering Committee, Dose Escalation and Data Review Intervals

The Trial Steering Committee (TSC) will comprise of at least four members and will include: Medical Monitor, one Principal Investigator, Pharmacokineticist and the Sponsor Representative. The Committee will make all decisions regarding dose escalations (or de-escalations) and assess achievement of the OBD. All decisions will be documented.

At the first stage of the study (dose escalation phase), the dose evaluation period will be from the first administration of begelomab up to Day 7 after last IMP administration in each cohort. There will be a minimum of 10 days between the Day 7 after the last IMP administration in one cohort and the first dose in the next cohort to allow adequate time for review of data.

The study is planned to comprise up to four dose levels; two additional dose levels may be added as needed. At the end of the dose escalation phase, the data from all cohorts will be analyzed to determine the optimal biologically active dose for further evaluation in the extended cohort. An interim Study Report including data collected during the dose escalation phase may be generated to require a scientific advice and for discussion with regulatory authorities.

At the second stage of the study (cohort extension), the accumulating efficacy and safety data will be continuously monitored by the TSC to ensure the risk-benefit profile remains favorable for the indicated population. The expansion cohort is planned to include up to 24 patients. The TSC can anyway decide to stop the enrollment earlier or extend accrual according to emerging results and eventual inputs from regulatory authorities.

### Dose Escalation Evaluation Process

Prior to each dose escalation, the TSC will meet to review all available safety, tolerability, PK and PD data. A decision to escalate the dose can only be made when none of the dose escalation stopping criteria (Section 6.2.4) are expected to be met at the next higher dose level.

A minimum of four evaluable subjects from each cohort are needed to reach a dose escalation decision; an ‘evaluable subject’ is defined as a subject that has completed all planned study activities as detailed in the protocol up to Day 7 after last dose of IMP.

### Dose Escalation Increments

Dose increments will be decided upon review of all available safety, tolerability, PK and PD data, taking into consideration the likelihood for non-linear pharmacokinetics. Dose increments will not exceed three-fold and will not be less than 30% higher than the dose level in the previous cohort.

The optimal biological dose is considered to be the dose leading to a maximum receptor occupancy, as determined by flow cytometry, and yielding the most desirable clinical effects.

### Prior and Concomitant Therapy

Prior medication refers to medication started within 30 days prior to screening and stopped prior to the first administration of the study treatment. Concomitant medication refers to medication started prior to and continued after the first administration of study treatment or taken any time after the first administration of the study treatment up to the follow-up visit.

Use of all concomitant medications will be recorded in the subject's CRF, regardless of whether they are permitted or prohibited medications, up to Study Day 57 or up to 30 days after the last dose of study treatment, whichever is later.

Any concomitant medication deemed necessary for the welfare of the subject during the study may be given at the discretion of the investigator. However, it is the responsibility of the investigator to ensure that details regarding the medication are recorded in full in the CRF. The minimum requirement is that the drug name, dose, indication and the dates of administration are recorded. Any changes in concomitant medications will also be recorded in the subject's CRF.

### Standard Therapy

Alongside the study treatments, the following drugs will be administered to subjects as baseline therapy:
methylprednisolone IV at the dose of ≤2 mg/kg/day (or other steroid and dose equivalent);
a calcineurin inhibitor (or other alternate, continuing GvHD prophylaxis).

They will be administered per the Principal Investigator's discretion and according to standard practice and institutional/international guidelines. Methylprednisolone or steroid equivalent dose may be escalated for worsening of acute GvHD. Steroid taper may follow local institutional practice. However, for those improving, steroid taper may not start sooner than 3 days after enrolment into the study and the steroid dose must not be tapered to less than 0.25 mg/kg/day prednisone (or 0.2 mg/kg/day methylprednisolone) on Day 28.

### Sequences

Heavy Chain Constant Region Amino Acid Sequence; Anti-CD26 mAb (begelomab)
SEQ ID NO: 1
AKTTPPSVYPLAPGCGDTTGSSVTLGCLVKGYFPESVTVTWNSGSLSSSVHTFPALLQSG
LYTMSSSVTVPSSTWPSQTVTCSVAHPASSTTVDKKLEPSGPISTINPCPPCKECHKCPA
PNLEGGPSVFIFPPNIKDVLMISLTPKVTCVVVDVSEDDPDVQISWFVNNVEVHTAQTQT
HREDYNSTIRVVSTLPIQHQDWMSGKEFKCKVNNKDLPSPIERTISKIKGLVRAPQVYIL
PPPAEQLSRKDVSLTCLVVGFNPGDISVEWTSNGHTEENYKDTAPVLDSDGSYFIYSKLN
MKTSKWEKTDSFSCNVRHEGLKNYYLKKTISRSPGK
Light Chain Constant Region Amino Acid Sequence; Anti-CD26 mAb (begelomab)
SEQ ID NO: 2
RADAAPTVSIFPPSSEQLTSGGASVVCFLNNFYPKDINVKWKIDGSERQNGVLNSWTDQD
SKDSTYSMSSTLTLTKDEYERHNSYTCEATHKTSTSPIVKSFNRNEC
Heavy Chain Variable Region (VH) Amino Acid Sequence; Anti-CD26 mAb (begelomab)
SEQ ID NO: 3
QVQLQQSGAELVKPGASVKLSCKASGYTFRSYDINWVRQRPEQGLEWIGWIFPGDGSTKY
NEKFKGKATLTTDKSSSTAYMQLSRLTSEDSAVYFCARWTVVGPGYFDVWGAGTTVTVSS
Heavy Chain Variable Region (VH) Nucleotide Sequence; Anti-CD26 mAb (begelomab)
SEQ ID NO: 4
caggtccagctgcagcagtctggagctgaactggtaaagcctggggcttcagtgaagttgtcctg
caaggcttctggctacaccttcagaagttatgatataaactgggtgagacagaggcctgaacagg
gacttgagtggattggatggatttttcctggagatggtagtactaagtacaatgagaagttcaag
ggcaaggccacactgactacagacaaatcctccagcacagcctacatgcagctcagcaggctgac
atctgaggactctgctgtctatttctgtgcaagatggacggtagtaggcccagggtacttcgatg
tctggggcgcagggaccacggtcaccgtctcctca
Light Chain Variable Region (VL) Amino Acid Sequence; Anti-CD26 mAb (begelomab)
SEQ ID NO: 5
QIVLTQSPAIMSASPGEKVTITCSASSSVSYMNWFQQKPGTSPKLWIYSTSNLASGVPAR
FSGSGSGTSYSLTISRMEAEDAATYYCQQRSSYPNTFGGGTKLEIK
Light Chain Variable Region (VL) Nucleotide Sequence; Anti-CD26 mAb (begelomab)
SEQ ID NO: 6
Caaattgttctcacccagtctccagcaatcatgtctgcatctccaggggagaaggtcaccataac
ctgcagtgccagctcaagtgtaagttacatgaactggttccagcagaagccaggcacttctccca
aactctggatttatagcacctccaacctggcttctggagtccctgctcgcttcagtggcagtgga
tctgggacctcttactctctcacaatcagccgaatggaggctgaagatgctgccacttattactg
ccagcaaaggagtagttacccgaacacgttcggaggggggaccaagctggaaataaaa
Heavy Chain CDR1 Amino Acid Sequence; Anti-CD26 mAb (begelomab)
SEQ ID NO: 7
GYTFRSYDIN
Heavy Chain CDR2 Amino Acid Sequence; Anti-CD26 mAb (begelomab)
SEQ ID NO: 8
WIFPGDGSTKYNEKFK
Heavy Chain CDR3 Amino Acid Sequence; Anti-CD26 mAb (begelomab)
SEQ ID NO: 9
WTVVGPGYFDV
Light Chain CDR1 Amino Acid Sequence; Anti-CD26 mAb (begelomab)
SEQ ID NO: 10
SASSSVSYMN
Light Chain CDR2 Amino Acid Sequence; Anti-CD26 mAb (begelomab)
SEQ ID NO: 11
STSNLAS
Light Chain CDR3 Amino Acid Sequence; Anti-CD26 mAb (begelomab)
SEQ ID NO: 12
QQRSSYPNT
Heavy Chain Constant Region Nucleotide Sequence; Anti-CD26 mAb (begelomab)
SEQ ID NO: 13
gccaaaacaacacccccatcagtctatccactggcccctgggtgtggagatacaactggttcctc
cgtgactctgggatgcctggtcaagggctacttccctgagtcagtgactgtgacttggaactctg
gatccctgtccagcagtgtgcacaccttcccagctctcctgcagtctggactctacactatgagc
agctcagtgactgtcccctccagcacctggccaagtcagaccgtcacctgcagcgttgctcaccc
agccagcagcaccacggtggacaaaaaacttgagcccagcgggcccatttcaacaatcaacccct
gtcctccatgcaaggagtgtcacaaatgcccagctcctaacctcgagggtggaccatccgtcttc
atcttccctccaaatatcaaggatgtactcatgatctccctgacacccaaggtcacgtgtgtggt
ggtggatgtgagcgaggatgacccagacgtccagatcagctggtttgtgaacaacgtggaagtac
acacagctcagacacaaacccatagagaggattacaacagtactatccgggtggtcagcaccctc
cccatccagcaccaggactggatgagtggcaaggagttcaaatgcaaggtcaacaacaaagacct
cccatcacccatcgagagaaccatctcaaaaattaaagggctagtcagagctccacaagtataca
tcttgccgccaccagcagagcagttgtccaggaaagatgtcagtctcacttgcctggtcgtgggc
ttcaaccctggagacatcagtgtggagtggaccagcaatgggcatacagaggagaactacaagga
caccgcaccagtcctggactctgacggttcttacttcatatatagcaagctcaatatgaaaacaa
gcaagtgggagaaaacagattccttctcatgcaacgtgagacacgagggtctgaaaaattactac
ctgaagaagaccatctcccggtctccgggtaaa
Light Chain Constant Nucleotide Sequence; Anti-CD26 mAb (begelomab)
SEQ ID NO: 14
cgggctgatgctgcaccaactgtatccatcttcccaccatccagtgagcagttaacatctggagg
tgcctcagtcgtgtgcttcttgaacaacttctaccccaaagacatcaatgtcaagtggaagattg
atggcagtgaacgacaaaatggcgtcctgaacagttggactgatcaggacagcaaagacagcacc
tacagcatgagcagcaccctcacgttgaccaaggacgagtatgaacgacataacagttatacctg
tgaggccactcacaagacatcaacttcacccattgtcaagagcttcaacaggaatgagtgt
Heavy Chain Nucleotide Sequence; Anti-CD26 mAb (begelomab-CHO optimized)
SEQ ID NO: 15
caagtgcagcttcagcagtccggagccgaactcgtgaagccgggagcttccgtgaagctgagctg
caaggcatcggggtataccttccgctcctacgacatcaactgggtcagacagaggcccgaacagg
gtctggaatggattggctggatcttccctggcgacggttccaccaagtacaacgagaagttcaag
ggcaaagccaccctgaccactgacaagtcctcatcgaccgcgtacatgcaattgagccggctgac
ctccgaggatagcgccgtgtacttctgtgcccggtggactgtcgtgggaccaggctactttgatg
tctggggggccggaactaccgtgacggtgtcatcagccaagactacccctccgtccgtgtacccc
cttgctccgggatgtggagacaccaccggctcgtccgtcactctgggatgcctcgtgaagggata
cttccccgaatccgtcaccgtgacctggaacagcggaagcctgtcctcgtccgtgcatactttcc
ctgccctgctgcaatccggcctgtacaccatgagctccagcgtgaccgtgccatcctcgacctgg
cccagccagaccgtgacttgctcagtggcgcaccctgcctcatccactaccgtggacaagaagct
cgagccctccggtccgatttcaaccatcaacccttgcccaccctgcaaagaatgccataagtgtc
ccgctccgaatctagaaggcggcccatccgtctttatcttccctcccaacattaaggacgtgctg
atgattagtctgaccccgaaagtcacttgcgtggtggtggacgtgtccgaagatgacccagacgt
gcagatctcatggttcgtgaacaacgtggaggtgcacacggcccagacccagacgcaccgggagg
actacaactcgactatccgcgtggtgtccacccttccgatccaacaccaggattggatgtcgggg
aaggagttcaagtgcaaggtcaacaacaaggatctcccgtcccccattgagaggacaatctctaa
gatcaagggcctcgtcagagcgcctcaggtctacatcttgcctcctcccgccgaacagttgagcc
ggaaggatgtgtccctgacttgtctggtcgtggggttcaatccgggagacatctccgtggagtgg
acctcgaacggacacaccgaggaaaactacaaggacactgcaccggtgctggattccgacggctc
ctatttcatctactcgaagctgaacatgaaaacctcgaaatgggaaaagactgacagcttcagct
gcaacgtgcgccacgagggtctgaagaactactacctgaaaaagaccatttcacggtccccgggg
aaa
Light Chain Nucleotide Sequence; Anti-CD26 mAb (begelomab-CHO optimized)
SEQ ID NO: 16
Cagatcgtgcttacccaatccccggcgattatgtcagccagccccggagaaaaggtcaccattac
ttgctcggcatcctcctccgtgtcgtacatgaactggttccagcaaaagcccggcactagcccaa
agctgtggatctattccacgtccaacctggcgtcaggagtgcctgcccgcttttcgggttctggc
agcgggactagctactccctcaccatctcgagaatggaagctgaggacgccgccacctactactg
tcagcagcggtcctcctacccgaacaccttcgggggaggcaccaaactggagatcaaacgggctg
atgctgcaccaactgtatccatcttcccaccatccagtgagcagttaacatctggaggtgcctca
gtcgtgtgcttcttgaacaacttctaccccaaagacatcaatgtcaagtggaagattgatggcag
tgaacgacaaaatggcgtcctgaacagttggactgatcaggacagcaaagacagcacctacagca
tgagcagcaccctcacgttgaccaaggacgagtatgaacgacataacagctatacctgtgaggcc
actcacaagacatcaacttcacccatcgtcaagagcttcaacaggaatgagtgt

1. A method of treating graft-versus-host disease (GvHD) in a subject, comprising administering a therapeutically effective amount of an anti-CD26 antibody to the subject, wherein the antibody is administered at a dose of between 4.0 mg/m²/day and 25.0 mg/m²/day, and wherein the antibody is administered once daily for five days followed by administration every other day for a total of 16 doses.

2. The method of claim 1, wherein the anti-CD26 antibody is a full-length antibody.

3. The method of claim 1, wherein the antibody is a monoclonal, human, humanized, chimeric, multivalent antibody, or an antigen-binding fragment thereof.

4. The method of claim 1, wherein the antibody has an isotype selected from the group consisting of IgG2, IgG3, IgG4, IgM, IgA, IgD and IgE.

5. The method of claim 4, wherein the antibody has an IgG2b isotype.

6. The method of claim 1, wherein the anti-CD26 antibody is begelomab, 1F7, or CM03.

7. The method of claim 6, wherein the anti-CD26 antibody is produced in Chinese hamster ovary (CHO) cells.

8. The method of claim 1, wherein the anti-CD26 antibody is produced from a hybridoma cell line deposited at CBA-ICLC of Genoa (Italy) as deposit number PD 12002.

9. The method of claim 1, wherein the wherein the anti-CD26 antibody comprises
(a) a heavy chain variable region CDR1 comprising the sequence set forth is SEQ ID NO:7;
(b) a heavy chain variable region CDR2 comprising the sequence set forth is SEQ ID NO:8;
(c) a heavy chain variable region CDR3 comprising the sequence set forth is SEQ ID NO:9;
(d) a light chain variable region CDR1 comprising the sequence set forth is SEQ ID NO:10;
(e) a light chain variable region CDR2 comprising the sequence set forth is SEQ ID NO:11; and
(f) a light chain variable region CDR3 comprising the sequence set forth is SEQ ID NO:12.

10. The method of claim 1, wherein the anti-CD26 antibody comprises heavy and light chain variable regions comprising the sequences set forth in SEQ ID Nos:3 and 5, respectively.

11. The method of claim 1, wherein the anti-CD26 antibody comprises heavy and light chains constant regions comprising the sequences set forth in SEQ ID Nos:1 and 2, respectively.

12. The method of claim 1, wherein the anti-CD26 antibody is administered at a dose of 4, 5, 6, 7, 8, 9, 10, 11, 12, 13, 14, 15, 16, 17, 18, 19, 20, 21, 22, 23, 24, or 25 mg/m².

13. The method of claim 12, wherein the anti-CD26 antibody is administered at a dose of 16 mg/m².
